(19) United States
(12) Patent Application Publication
(10) Pub. No.: US 2014/0026300 A1
(43) Pub. Date: Jan. 30, 2014

(54) AIRFLOW BARRIER SYSTEM FOR PROTECTIVE EYEWEAR

(71) Applicant: Ricardo Silveira, Bridgewater, MA (US)

(72) Inventor: Ricardo Silveira, Bridgewater, MA (US)

(73) Assignee: HONEYWELL INTERNATIONAL, INC., Morristown, NJ (US)

(21) Appl. No.: 13/560,652

(22) Filed: Jul. 27, 2012

(51) Int. Cl. A61F 9/00

(52) U.S. Cl. 2/431

(57) ABSTRACT

A protective eyewear includes a frame, a lens supported on the frame, and an air delivery system including a nozzle adjacent to an outer surface of the lens. The nozzle conforms to the contour of the lens and is configured and arranged to direct a high-speed flat, laminar airflow over the outer surface of the lens whereby the airflow reduces the impact and collection of airborne contaminants on the outer surface of the lens.

### BACKGROUND

The instant invention relates to protective eyewear and more particularly to an active airflow barrier system for delivering a high-speed, flat laminar flow of air across the outer surface of a lens of the eyewear. The focused airflow creates an active, invisible protective barrier that reduces the impact and collection of airborne contaminants, such as dirt, dust, mist and spray, on the outer surface of the lens which would otherwise impede the wearer's visibility.

### SUMMARY

An improved protective eyewear generally includes an eyewear frame, a lens supported on the frame, and an air delivery system including a laminar airflow nozzle immediately adjacent to an outer surface of the lens. The nozzle conforms to the contour of the lens and is configured and arranged to direct a high-speed flat, laminar airflow over the outer surface of the lens whereby the airflow creates an active, invisible protective barrier to reduce the impact and collection of airborne contaminants on the outer surface of the lens.

The exemplary embodiment is described as a welding helmet having a lens supported in the front wall thereof. While the exemplary embodiment is a welding helmet, it is to be understood that the concepts disclosed herein are equally applicable to all forms of protective eyewear that include a lens, including but not limited to safety glasses, goggles, respiratory masks and face shields.

The air delivery system generally includes a fan for creating a pressurized high-speed airflow, a laminar airflow nozzle, and a conduit connecting the fan and the nozzle to convey the airflow from the fan to nozzle. The fan is a portable electric fan system including a portable DC power source and control circuit for selectively controlling the speed and volume of airflow. The fan system is integrated into a wearable belt assembly which is carried by the end user during operation. The exemplary embodiment of the conduit is flexible hose connecting the outlet of the fan to an input of the laminar airflow nozzle.

In the exemplary embodiment, the laminar airflow nozzle is integrated into the top portion of the welding helmet frame above the lens and is configured and arranged to convert the airflow from the conduit into a high-speed, flat, laminar air flow which is directed downwardly over the lens. While the exemplary embodiment, discloses an integrated nozzle construction, it should be noted that the nozzle could be configured as a separate component which is removably attached to the protective eyewear.

In operation, the fan system generates a high-speed pressurized airflow which is conveyed through the conduit to the nozzle which simultaneously converts the high-speed airflow to a high-speed flat, laminar airflow, and directs the high-speed, flat, laminar airflow downwardly over the outer surface of said lens. More specifically, the nozzle includes a reduced dimension exit orifice having a narrow, elongated shape which conforms to a contour of the outer surface of the lens. As indicated above, the flat, laminar airflow creates an active, invisible protective barrier to reduce the impact and collection of airborne contaminants on the outer surface of the lens.

In an alternate embodiment, the airflow nozzle is located on a side portion of welding helmet frame, and the high-speed, flat laminar airflow is directed in a side-to-side direction over the outer surface of the lens.

Accordingly, it is an objective of the present disclosure to provide a protective eyewear which includes an air delivery system effective for creating active, invisible protective air barrier to reduce the impact and collection of airborne contaminants on the outer surface of the eyewear lens.

It is another objective to provide a protective eyewear including a laminar flow nozzle adjacent to the lens which directs a high-speed, laminar air flow over the surface of the eyewear lens.

Other objects, features and advantages shall become apparent as the description thereof proceeds when considered in connection with the accompanying illustrative drawings.

### BRIEF DESCRIPTION OF THE DRAWING FIGURES

An exemplary embodiment will now be described by way of example with reference to the following figures which are intended to be illustrative only and in no way limiting upon the scope of the disclosure:

FIG. 1 is a perspective view of the protective eyewear in accordance with the present disclosure;

FIG. 2 is a side view thereof; and

FIG. 3 is a perspective view of an alternative embodiment.

### DETAILED DESCRIPTION OF AN EXEMPLARY EMBODIMENT

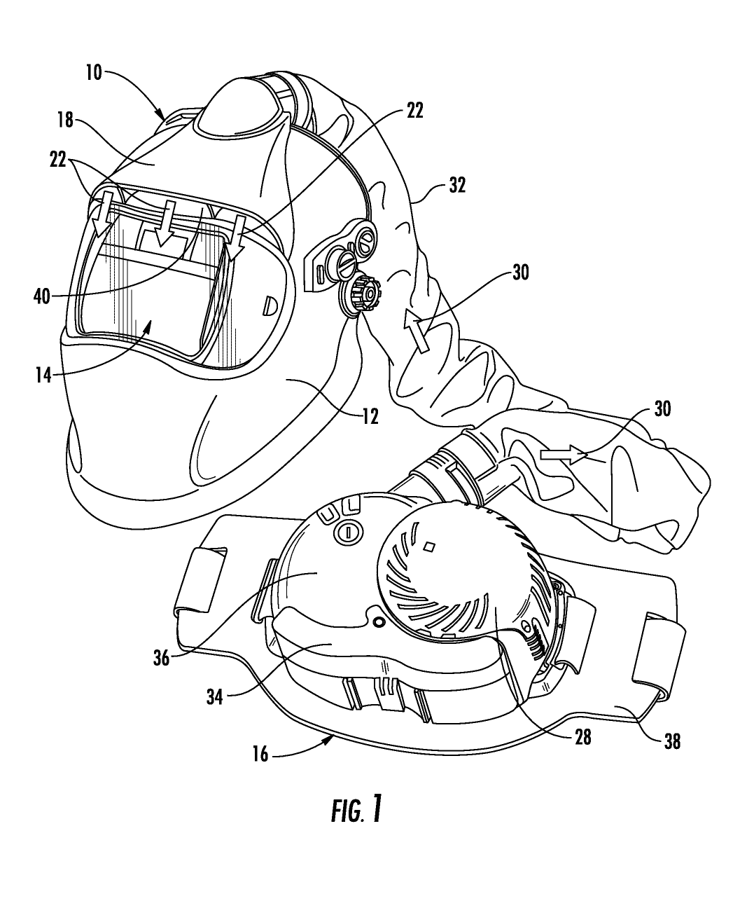

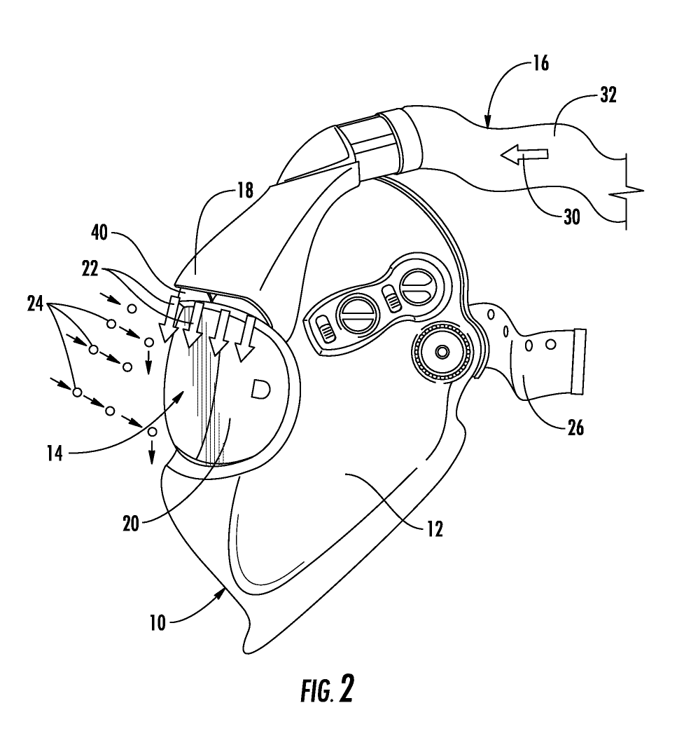

Referring to FIGS. 1 and 2, an improved protective eyewear 10 generally includes an eyewear frame 12, a lens 14 supported on the frame 12, and an air delivery system 16 including a laminar airflow nozzle 18 immediately adjacent to an outer surface 20 of the lens. As will be described further herein below, the nozzle 18 conforms to the contour of the lens 14 and is configured and arranged to direct a high-speed flat, laminar airflow 22 over the outer surface 20 of the lens 14 whereby the airflow 22 creates an active, invisible protective barrier to reduce the impact and collection of airborne contaminants 24 on the outer surface 20 of the lens 14.

As illustrated in the drawing Figures, the exemplary embodiment of the protective eyewear 10 is a welding helmet having lens 14 supported in the front wall thereof. Welding helmets of the type contemplated also include a headgear 26 for supporting the helmet frame 12 on the head of the end user (see FIG. 2). While the exemplary embodiment is described as a welding helmet, it is to be understood that the concepts disclosed herein are equally applicable to all forms of protective eyewear that include a lens, including but not limited to safety glasses, goggles, respiratory masks and face shields.

The air delivery system 16 generally includes a fan 28 for creating a pressurized high-speed airflow 30, and a conduit 32 connecting the fan 28 to the nozzle 18 to convey the airflow 30 from the fan 28 to the nozzle 18. The fan 28 is a portable electric fan system including a portable DC power source 34, such as a rechargeable battery, and control system 36 for selectively controlling the speed and volume of airflow 30. The fan system 28 is integrated into a wearable belt assembly 38, or other type of assembly which can be worn or carried by the end user during operation. The exemplary embodiment of the conduit 32 is flexible hose connecting the outlet of the fan 28 to an inlet of the laminar airflow nozzle 18.

In the exemplary embodiment, the laminar airflow nozzle 18 is integrated into the top portion of the welding helmet frame 12 above the lens 14 and is configured and arranged to convert the airflow 30 from the conduit 32 into a high-speed, flat, laminar airflow 22 which is directed downwardly over the lens 14. While the exemplary embodiment, discloses an integrated nozzle construction, it should be noted that the nozzle 18 could be configured as a separate component which is removably attached to the protective eyewear 10.

In operation, the fan system 28 generates a high-speed pressurized airflow 30, which is conveyed through the conduit 32 to the nozzle 18 which simultaneously converts the high-speed airflow 30 to a high-speed flat, laminar airflow 22, and directs the high-speed, flat, laminar airflow 22 downwardly over the outer surface 20 of the lens 14. More specifically, the nozzle 18 includes a reduced width exit orifice 40 generally having a narrow, elongated shape which conforms to a contour of the outer surface 20 of the lens 14 so that the laminar airflow 22 is directed in close proximity to the outer surface 20 of the lens 14. As indicated above, the flat, laminar airflow 22 travelling at a high rate of speed over the lens surface 20 creates an active, invisible protective barrier to reduce the impact and collection of airborne contaminants 24 on the outer surface 20 of the lens 14.

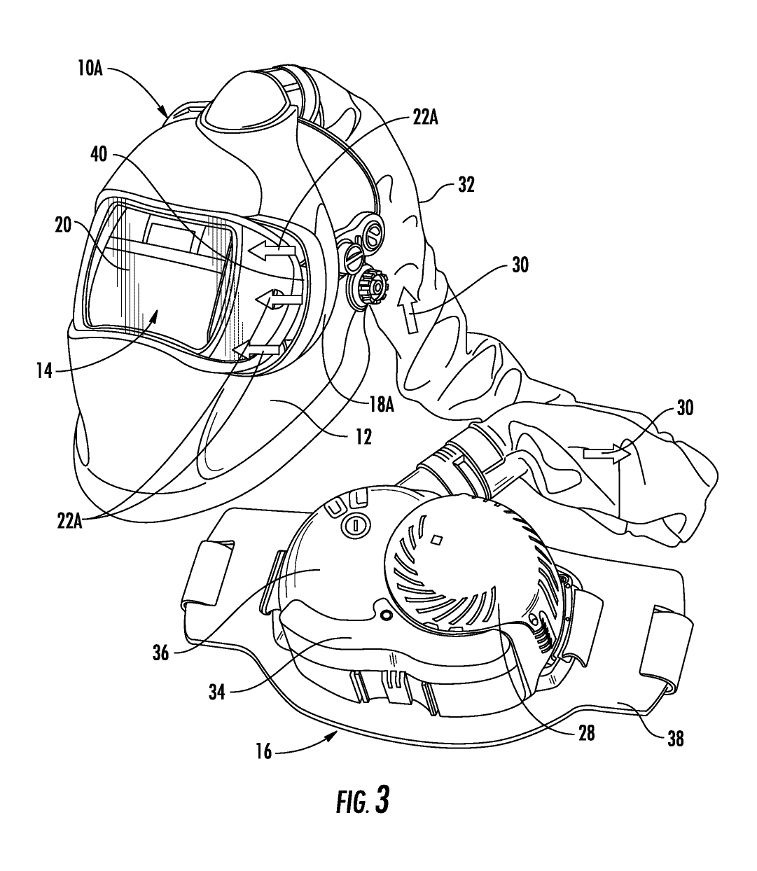

In an alternate embodiment 10A as illustrated in FIG. 3, the airflow nozzle 18A is located on a side portion of welding helmet frame 12, and the high-speed, flat laminar airflow 22A is directed in a side-to-side direction over the outer surface 20 of the lens 14 with the same effect.

It can therefore be seen that the present disclosure provides a protective eyewear 10 which includes an air delivery system 16 effective for creating active, invisible protective air barrier to reduce the impact and collection of airborne contaminants on the outer surface of the eyewear lens. For these reasons, the present disclosure is believed to represent a significant advancement in the art which has substantial commercial merit.

While there is shown and described herein certain specific structure, it will be manifest to those skilled in the art that various modifications and rearrangements of the parts may be made without departing from the spirit and scope of the underlying concepts and that the same is not limited to the particular forms herein shown and described, except insofar as indicated by the scope of the appended claims.

What is claimed is:

1. A protective eyewear comprising:
a frame;
a lens supported on said frame; and
an air delivery system including a nozzle adjacent to an outer surface of said lens,
said nozzle directing a flat, laminar airflow over said outer surface of said lens,
said airflow reducing impacts of airborne contaminants on said outer surface of said lens.

2. The protective eyewear of claim 1 wherein said nozzle is located on a top portion of said frame, and said airflow is directed in a downward direction over said outer surface of said lens.

3. The protective eyewear of claim 1 wherein said nozzle is located on a side portion of said frame, and said airflow is directed in a side-to-side direction over said outer surface of said lens.

4. The protective eyewear of claim 1 wherein said nozzle includes an exit orifice having a narrow, elongated shape which conforms to a contour of said outer surface of said lens.

5. The protective eyewear of claim 2 wherein said nozzle includes an exit orifice having a narrow, elongated shape which conforms to a contour of said outer surface of said lens.

6. The protective eyewear of claim 3 wherein said nozzle includes an exit orifice having a narrow, elongated shape which conforms to a contour of said outer surface of said lens.

7. The protective eyewear of claim 1 wherein said nozzle is integrally formed as part of said frame.

8. The protective eyewear of claim 2 wherein said nozzle is integrally formed as part of said frame.

9. The protective eyewear of claim 3 wherein said nozzle is integrally formed as part of said frame.

10. A protective eyewear comprising:
a frame;
a lens supported on said frame;
a fan;
a nozzle adjacent to an outer surface of said lens; and
a conduit connecting said fan and said nozzle, said conduit conveying an airflow from said fan to said nozzle,
said nozzle converting said airflow from said fan into a high-speed, flat, laminar airflow,
said nozzle directing said high-speed, flat, laminar airflow over said outer surface of said lens,
said high-speed, flat, laminar airflow reducing impacts of airborne contaminants on said outer surface of said lens.

11. The protective eyewear of claim 10 wherein said nozzle is located on a top portion of said frame, and said high-speed, flat laminar airflow is directed in a downward direction over said outer surface of said lens.

12. The protective eyewear of claim 10 wherein said nozzle is located on a side portion of said frame, and said high-speed, flat laminar airflow is directed in a side-to-side direction over said outer surface of said lens.

13. The protective eyewear of claim 10 wherein said nozzle includes an exit orifice having a narrow, elongated shape which conforms to a contour of said outer surface of said lens.

14. The protective eyewear of claim 11 wherein said nozzle includes an exit orifice having a narrow, elongated shape which conforms to a contour of said outer surface of said lens.

15. The protective eyewear of claim 12 wherein said nozzle includes an exit orifice having a narrow, elongated shape which conforms to a contour of said outer surface of said lens.

16. The protective eyewear of claim 10 wherein said nozzle is integrally formed as part of said frame.

17. The protective eyewear of claim 11 wherein said nozzle is integrally formed as part of said frame.

18. The protective eyewear of claim 12 wherein said nozzle is integrally formed as part of said frame.
